(19) United States
(12) Patent Application Publication
(10) Pub. No.: US 2005/0054991 A1
(43) Pub. Date: Mar. 10, 2005

(54) Topical administration device

(76) Inventors: Michael John Tobyn, Wiltshire (GB); John Nicholas Staniforth, Bath (GB); Sharon Ann Mills, Bath (GB); Graeme Lindsay Jonathan Paterson, Somerset (GB); Christopher Althorpe, Cardiff (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/488,169

(22) PCT Filed: Aug. 28, 2002
(86) PCT No.: PCT/IB02/03485

(30) Foreign Application Priority Data
Aug. 29, 2001 (US) 60315894

(51) Int. Cl. A61M 35/00

(52) U.S. Cl. 604/290; 604/289

(57) ABSTRACT

Disclosed is a dosing device for topically administering a pharmaceutical formulation to a skin of a mammal, the device comprising a housing capable of holding at least one unit dose of a pharmaceutical formulation comprising a drug and a carrier; an applicator adapted for topically administering a unit dose of the pharmaceutical formulation directly onto the skin; and an actuator, wherein upon actuation, the device is capable of metering a unit dose of the pharmaceutical formulation external to the device, from the housing to the applicator; and methods thereof.

### FIELD OF INVENTION

The present invention is directed to a dosing device, which can be utilized to meter and administer a pharmaceutical formulation to the skin of a mammal, e.g., humans, and methods thereof.

### BACKGROUND OF THE INVENTION

Drug therapy prescribed by a health care professional typically includes the selection of drug, the potency or strength of the drug and the appropriate dosing interval. Most pharmaceutical formulations, e.g., tablets, capsules and liquids can meet these requirements as a patient can take a unit dose of the prescribed drug which has been precalibrated to provide an indicated strength. An oral liquid formulation when used correctly gives even more precision as the liquid can be measured with a standard measurer in order to obtain precise individual doses of drug. When a prescriber diagnosis a condition and prescribes a topical treatment such as a cream, ointment, lotion, liquid, etc, precision dosing is more difficult.

Creams and ointments are typically packages in a tube or jar and exact dosing of drug cannot be calibrated by the patient. In fact, many times, the prescriber simply instructs the patient to apply a particular formulation a certain number of times daily, e.g. twice daily, and does not provide the patient with any insight as to what would be too little of a dose (sub-therapeutic) or what would be too much (possibly resulting in increased side effects).

Another problem associated with topical pharmaceutical formulations is that when a topical formulation is dispensed from a tube, the subsequent administration is typically by the patient or caregiver taking the formulation in their hand and applying it to the affected area. This method has many undesirable consequences. For example, the amount dispensed from the tube is not the amount, which will be administered to the intended site of action. This is due to the fact that an amount of the formulation will be absorbed into the skin of the hand of the patient or the caregiver. Therefore, even assuming that the patient fortuitously dispenses a proper amount of formulation, a subtherapeutic dose may be applied due to the amount absorbed by the skin. This can be avoided by, e.g., the patient wearing a latex glove during application. This is often objectionable for many reasons. Many people find the feel of latex gloves unacceptable and uncomfortable. Also, many people are allergic to latex and the use of such an administration aid can precipitate anaphylactic shock. Further, latex gloves add additional expense to drug therapy and may not be readily available to all patients.

Another problem of the hand administration method is that drug can be absorbed to an area that is not intended to be. For example, if the hand is used to apply formulation prescribed for the torso, drug will also be absorbed by the hand. This can be a problem with drugs which have high toxicity or produce undesirable side effects. Another issue with this common form of administration is that many patients do not take appropriate measure to clean and sanitize their hands prior to administration which can lead to spread of microbes.

The prior art is replete with specific examples of topical formulations where specific dosing regimens, and/or particular maximum dosages, have been required. For example, doxepin hydrochloride, a systemic antidepressant agent, is recommended to be applied thinly three to four times daily, typically with a maximum 3 g administration per application, a typical daily maximum administration of 12 g and suitably coverage should be less than about 10% of body surface.

Clobetasol propionate, a steroidal anti-inflammatory agent is recommended to be applied thinly one to two times daily for up to four weeks, typically with a maximum administration of about 50 g of a 0.05% preparation per week.

Diflucortolone valerate, another anti-inflammatory agent is recommended to be applied one to two times daily for up to four weeks (0.1% preparation) or two weeks (0.3% preparation), with typically a maximum administration of about 60 g of a 0.3% preparation per week.

Monitoring the maximum dosage has been especially important for topical formulations of calcipotriol. There has been seen to be a risk of hypercalcemia if the recommended maximum weekly dose of calcipotriol has been exceeded. The risk of hypercalcemia and methods to avoid this risk have not always been clearly explained in patient information provided with topical formulations of calcipotriol. For example, the package insert for the topical formulation of calcipotriol available under the trademark Dovonex, advises liberal application of the formulation despite the above described disadvantage of possible hypercalcemia with excessive dosing. The recommended dosing regime for a topical formulation of calcipotriol, however, has been to apply once or twice daily, with a maximum weekly dose of 100 g. For patients over six years of age, the formulation should be applied twice daily; for patients from six to twelve years, a maximum weekly dose of 50 g; and for patients over twelve years, a maximum weekly dose of 75 g.

To alleviate problems encountered with treatment regimes where it has been important to observe a maximum dosage of a therapeutic agent for topical administration, it would be beneficial to be able to provide means for accurately administering a therapeutic agent and applying it directly to the skin of a patient without the need for intermediary manipulation by the patient or the caregiver. Such accurate administration should obviate the detrimental side effects that have hitherto been observed.

With respect to the inaccuracy of dosing associated with typical topical formulation tubes, one method of metering, or dosing, the amount of a therapeutic agent applied to a patient's skin in such a topical formulation has been for a patient to squeeze such a topical formulation from a dispenser, such as a tube, along an index finger starting at the fingertip down to the first joint and the amount of therapeutic agent thus to be administered has been known as the fingertip unit (FTU). One FTU generally approximates to about 500 mg of a topical formulation and is generally sufficient to cover an area that is twice that of a flat adult hand. Such administration has not, however, hitherto achieved accurate dosing. In particular, a significant disadvantage associated with the FTU is that it is only an approximate unit and its magnitude varies from patient to patient.

Another method, as in the case of nitroglycerin paste, is to measure the paste on a calibrated paper supplied by the manufacturer. This is also not accurate as the width of the paste being measured can significantly alter the measurement. For example, if the patient dispenses the ointment slowly, a wider mass will be measured longitudinally, which would result in an overdose. Conversely, if the patient dispenses over the measured area quickly, the width of the paste being measured may be thinner and a subtherapeutic dose may result.

It has also been known to deliver therapeutic agents transdermally by applying to the skin of a patient an adhesive patch containing a therapeutic agent. Such patches have typically further included a rate-moderating membrane, an adhesive, a liner and a backing material. The adhesive has often required special formulation to ensure compatibility with the other components of such patches and this type of formulation has often increased the cost of such patches. Furthermore, not all therapeutic agents are suitable for inclusion in such patches for many reasons, such as stability, absorption, etc.

Due to the disadvantages of topical pharmaceutical formulations discussed above, there exists a need in the art for the development of a device and method which address both the problem of inaccurate dosing and the problem of administration as discussed above.

### OBJECTS AND SUMMARY OF THE INVENTION

It is an object of the present invention to provide a dosing device for the topical administration of a unit dose of a pharmaceutical formulation

It is a further object of certain embodiments of the invention to provide a dosing device for containing a supply of multiple unit doses of pharmaceutical formulation, the device capable of metering an accurate unit dose of the pharmaceutical formulation.

It is a further object of certain embodiments of the invention to provide a dosing device for containing a single unit dose of pharmaceutical formulation, the device capable of dispensing and applying the unit dose onto the skin of a patient.

It is a further object of certain embodiments of the invention to provide a dosing device for containing a supply of multiple unit doses of pharmaceutical formulation, the device capable of dispensing and applying the formulation onto the skin of a patient.

It is a further object of certain embodiments of the invention to provide a dosing device for containing a supply of multiple unit doses of pharmaceutical formulation, the device capable of metering an accurate unit dose of the pharmaceutical formulation and applying the unit dose onto the skin of a patient.

It is a further object of certain embodiments of the invention to provide a dosing device for containing a supply of multiple unit doses of pharmaceutical formulation, the device capable of metering an accurate unit dose of the pharmaceutical formulation and applying the unit dose onto the skin of a patient wherein the device can be operated with one hand.

It is a further object of certain embodiments of the invention to provide a dosing device for containing a supply of multiple unit doses of pharmaceutical formulation, the device capable of metering an accurate unit dose of the pharmaceutical formulation and applying the unit dose onto the skin of a patient without the need for the patient or caregiver having to apply the drug manually or with an intermediate receptacle.

It is a further object of certain embodiments of the invention to provide a dosing device for containing a supply of multiple unit doses of pharmaceutical formulations, the device capable of use by a caregiver without exposing themselves to the drug, e.g., their hands.

It is a further object of certain embodiments of the invention to provide a dosing device for containing a supply of multiple unit doses of pharmaceutical formulations, the device capable of use by a patient without exposing themselves to the drug at an undesired location, e.g., their hands.

The above objects of the invention and others, can be achieved by virtue of the present invention which in certain embodiments is directed to a dosing device for topically administering a pharmaceutical formulation to the skin of a mammal, the device comprising a housing capable of storing at least one unit dose of a pharmaceutical formulation comprising a drug incorporated with a pharmaceutically acceptable carrier suitable for topical application onto the skin of said mammal; an applicator adapted for topically administering a unit dose of the pharmaceutical formulation directly onto the skin; and an actuator capable of metering a single unit dose of the pharmaceutical formulation from a first position in which the unit dose is stored in the housing to a second position in which the single unit dose is external to the device on the applicator so that the single unit dose can be topically administered

In other embodiments, the invention provides a dosing system comprising a pharmaceutical formulation comprising a drug and a carrier suitable for topical application contained in a dosing device for topically administering a pharmaceutical formulation to the skin of a mammal, the device comprising a housing capable of storing at least one unit dose of a pharmaceutical formulation comprising a drug incorporated with a pharmaceutically acceptable carrier suitable for topical application onto the skin of said mammal; an applicator adapted for topically administering a unit dose of the pharmaceutical formulation directly onto the skin; and an actuator capable of metering a single unit dose of the pharmaceutical formulation from a first position in which the unit dose is stored in the housing to a second position in which the single unit dose is external to the device on the applicator so that the single unit dose can be topically administered.

In other embodiments, the invention is directed to a method for topically administering a pharmaceutical formulation to the skin of a mammal, the method comprising (i) actuating a dosing device for topically administering a pharmaceutical formulation to the skin of a mammal, the device comprising a housing storing at least one unit dose of a pharmaceutical formulation comprising a drug incorporated with a pharmaceutically acceptable carrier suitable for topical application onto the skin of said mammal; an applicator adapted for topically administering a unit dose of the pharmaceutical formulation directly onto the skin; and an actuator capable of metering a single unit dose of the pharmaceutical formulation from a first position in which the unit dose is stored in the housing to a second position in which the single unit dose is external to the device on the applicator so that the single unit dose can be topically administered; and (ii) applying the unit dose directly onto the skin of a mammal with the applicator.

In other embodiments, the invention is directed to a method of preparing a dosing system for topical delivery of a pharmaceutical formulation including (i) preparing at least one unit dose of a pharmaceutical preparation comprising a drug incorporated with a pharmaceutically acceptable carrier suitable for topical application onto the skin of said mammal; and (ii) placing the at least one unit dose into a dosing device comprising a housing capable of storing at least one unit dose of a pharmaceutical formulation comprising a drug incorporated with a pharmaceutically acceptable carrier suitable for topical application onto the skin of said mammal; an applicator adapted for topically administering a unit dose of the pharmaceutical formulation directly onto the skin; and an actuator capable of metering a single unit dose of the pharmaceutical formulation from a first position in which the unit dose is stored in the housing to a second position in which the single unit dose is external to the device on the applicator so that the single unit dose can be topically administered. The term “semi-solid” for purposes of the present invention includes ointments, gels, emulsion, mousse, magmas, milks, pastes, creams and foams. In certain preferred embodiments of the present invention, the semi-solid is an ointment, cream or gel.

For purposes of the present invention, the term “device” refers to an apparatus capable of delivering at least one unit dose of drug.

The term “system” refers to a drug delivery device in combination with a pharmaceutical formulation for topical delivery.

The term “therapeutic agent” or “drug” as used herein denotes any active substance suitable to be topically administered to a mammal, e.g., humans) for a therapeutic or prophylactic purpose and being suitable for use in any formulation in connection with the present invention. The term “therapeutic agent” as used herein also includes any pharmaceutically acceptable equivalent of the active substance, such as a pharmaceutically acceptable salt, ester, prodrug or metabolite thereof. Isomers of all disclosed agents are also encompassed by this disclosure.

The term “unitary” when used with respect to the device of the present invention means that the applicator and the housing are in a fixed position and do not have to be removed from each other to apply the unit dose of formulation from the applicator, or have their orientation with respect to each other altered in order to apply the dose. It is preferred that the spatial relationship between the housing and the applicator are the same before, during, and after actuation and subsequent application.

The terms “topically administered” or “topical administration” as used herein includes (i) administration of a therapeutic agent suitable for use in the present invention for local treatment on the surface of the skin; (ii) administration of a therapeutic agent which is absorbed to provide a local effect in the region of application (e.g., in the muscle or tissue at or near the point of administration; and (iii) administration of a therapeutic agent suitable for use in the present invention for non-local treatment by administration through the skin, in other words for administration into the blood stream of a mammal for systemic treatment.

The term “treatment” as used herein denotes the treatment of established conditions as well as the prophylaxis thereof. The precise treatment conditions for any pharmaceutical formulation, product or method according to the present invention will of course depend on the precise nature of a condition being treated, the age and sex of the patient and will ultimately be at the discretion of an attendant physician.

The term “drug” refers to any agent which is capable of providing a therapeutic effect to a patient

The term “dispense”, when used in connection with the devices and systems of the present invention, means that the device or system delivers a unit dose contained in the housing of the device to the applicator, external from the device.

The term “administer”, when used in connection with the devices and systems of the present invention, means that the device applies the unit dose directly onto the skin

The term “patient” refers to humans as well as other mammals in need of a topical therapeutic agent, e.g., household pets or livestock. This term also refers to humans or mammals in need of or receiving prophylactic treatment.

The term “unit dose” means a formulation suitable for single administration which contains an effective amount of an agent to be administered.

### BRIEF DESCRIPTION OF THE DRAWINGS

FIG. 1 is a view of one embodiment of a dosing device according to the present invention.

FIG. 2 is a section view of the embodiment shown in FIG. 1.

FIG. 3 is an exploded view of the components of the embodiment shown in FIG. 1.

FIG. 4a is a partial section view of the embodiment shown in FIG. 1 showing the applicator in a closed position.

FIG. 4b is a partial section view of the embodiment shown in FIG. 1 showing the applicator in an open position.

FIGS. 5a, 5b, and 5c are perspective view of a button mechanism of the embodiment shown in FIG. 1.

FIG. 6 is a perspective view of a button non-return mechanism of the embodiment shown in FIG. 1.

### DETAILED DESCRIPTION

The present invention relates to a dosing device for topically administering a pharmaceutical formulation directly to the skin of a mammal. The dosing device of the present invention includes a housing capable of holding at least one unit dose of a pharmaceutical formulation comprising a drug and a suitable carrier therefor, an applicator adapted for topically administering the unit dose of a pharmaceutical formulation directly onto the skin, and an actuator. When the dosing device is actuated, the device can meter a unit dose of the pharmaceutical formulation from the housing to the applicator.

The dosing device of the invention can be used to apply a pharmaceutical formulation directly to the skin of a mammal e.g., as a semi-solid or liquid pharmaceutical formulation. Certain embodiments of the invention are adapted to contain and meter semi-solid pharmaceutical formulations such as an ointment, gel, emulsion, lotion, spray, cream or paste and certain embodiments are adapted to contain and meter a liquid such as a suspension or solution.

The pharmaceutical formulation can be placed in the housing of the dosing device, wherefrom unit doses can be metered directly to the applicator and administered to the skin according to a dosing schedule either by the patient or the caregiver. The size of the unit dose is dependent on the amount of drug to be provided for the intended therapeutic effect and the amount of the pharmaceutically acceptable carrier medium. Typically, a unit dose from about 0.10 grams to about 5 grams can be metered from the housing to the applicator and would be sufficient to contain a therapeutically effective amount of the drug to be delivered. However, this range is not limiting and can be smaller or higher, depending on the amount or potency of drug and carrier that is necessary. Additional unit doses can be delivered from the housing to the applicator upon a subsequent actuation of the actuator until depletion of the pharmaceutical formulation from the housing.

In order to promote patient compliance, certain embodiments of the invention include a counter which indicates the number of doses actuated. Alternatively, the dosing device can include an indicator to display the number of doses remaining in the dosing device. The ability to count remaining doses is useful especially to a patient who may have forgotten if a previous dose has been taken. A counter also minimizes the likelihood of the patient miscounting the proper dosage and taking a double dose or skipping a dose due. The counter will also keep the user apprized as to when the drug will run out and will help to improve patient compliance by allowing for proper planning for the patient to frequent a pharmacy in a timely manner. This can reduce the likelihood of a patient being “surprised” when the system does not provide any unit doses. The device can alternatively count the doses delivered by counting up, or can count down to show the number of unit doses remaining in the system. The counter can be an electrical or mechanical mechanism which are commonly known in the art. The indicator can also be a visual mechanism, e.g., the topical formulation could fall below a colored marker which would indicate the number of doses remaining, the device can expose the internal formulation to view in a window, or other mechanisms known in the art.

In certain embodiments, after depletion or partial depletion of the at least one unit dose, the dosing device can optionally be reloaded with at least one additional unit dose. Alternatively, if the dosing device is not capable of being reloaded, then the device is disposable. This embodiment is beneficial for many reasons. Most prominently, a disposable device will give a patient, the prescriber and the manufacturer greater assurances that the patient is receiving a proper dosage from a dosing system that has not been subject to improper handling an/or internal handling for a long duration of time. Such disposable devices may also reduce the overall cost of manufacture, as the device would only have to be manufactured to provide an accurate dosage for a finite period of time.

The housing of the device is preferably capable of containing multiple doses of the pharmaceutical formulation in order to provide a multiplicity of unit doses. The number of unit doses contained in the housing of the device and capable of being delivered onto the skin depends on, among other factors, the frequency of dosing and the duration of therapy of the drug to be dispensed. Preferably, the housing of the dosing device can hold from about 5 unit doses to about 400 unit doses of the pharmaceutical formulation. More preferably, the housing of the dosing device is adapted to contain from about 40 doses to 120 unit doses of the pharmaceutical formulation. In certain aspects of the invention, the housing of the dosing device is preferably adapted to contain at least 2 unit doses, and more preferably at least 5 unit doses of the pharmaceutical formulation. In other aspects, the housing of the dosing device can preferably contain 30 unit doses, and more preferably 365 unit doses.

In certain embodiments, the dosing device can contain multiple dosing mechanisms in order to provide dosage amounts for different times. For example, the system can comprise two dosing mechanisms which can provide a different dosage amount in the morning and the evening.

In certain embodiments, the dosing device of the invention can include more than one housing, each containing a different drug or pharmaceutical formulation. Upon actuation, the desired amount of each drug is metered out for delivery to the skin at the same time or sequentially as desired. Dosing devices containing multiple housings or multiple reservoirs in a housing would be beneficial for combination therapy, would eliminate the need for multiple devices and would allow a much wider range of possible doses and dose combinations.

In certain embodiments, the system of the invention can be configured wherein the housing is replaceable, e.g., in the form of a replaceable cartridge, or wherein the housing is capable of being refilled, e.g., by including a removable plug wherein bulk topical formulation can be introduced. However, in embodiments wherein the housing is capable of being refilled, it is preferable that a replaceable housing is utilized such as the previously disclosed cartridge device rather than refilling the housing with a bulk formulation through an unplugged hole as the latter may be more prone to human error, e.g., loss of product due to spilling or improper manipulation. Further, the handling of bulk topical formulation may result in contamination of the device, formulation or both, with moisture and/or contaminants.

In certain preferred embodiments, the dosing device is ergonomically engineered to facilitate a caregiver to administer a topical pharmaceutical formulation to the skin of a patient and in other embodiments the dosing device is ergonomically engineered to facilitate self-administration. Preferably, the dosing device is ergonomically engineered to facilitate both situations.

In certain preferred embodiments, the dosing device of the invention could include a means for preventing the actuator from functioning after a predetermined number of actuation, for the predetermined time period. The means controlling the function of the actuator can be mechanical or electrical means as known in the art. The dosing device of the invention can be configured such that the desired time period is the dosing interval of the drug. For example, in certain preferred embodiments, the dosing interval of the dosing device is from about one hour to about twenty-four hours, more preferably from about four (4) hours to about twelve (12) hours. In certain embodiments, the dosing device is engineered such that the predetermined number of actuations is a number which administers the prescribed amount of a pharmaceutical formulation, preferably such that the predetermined number of actuations is one actuation or more preferably is more than one actuation. Moreover, in order to prevent accidental actuation during application of the dosing device to the skin, in certain aspects, the desired time period for preventing the actuator from functioning is at least the time needed to administer topically the previously metered unit dose.

In certain embodiments, the pharmaceutical composition included in the device does not exceed a 10% overage, preferably does not exceed a 5% overage and most preferably does not require any overage. In situations where there is overage, the device can be configured with a mechanism which prevents the patient from accessing the overage. This could prevent the patient from being administered a partial dose which may be subtherapeutic. This feature can also prevent a patient from being administered more unit doses than prescribed by the physician.

It is important that the dosing device of the invention provides accurate and reproducible unit doses. Accordingly, in certain embodiments, each unit dose metered from the device does not vary by more than 10% throughout the life of the device at room and other temperature ranges. The temperature range may vary from 0° C. to less than 100° C., more preferably from about 10° C. to about 80° C., and most preferably from about 20° C. to about 40° C. In certain other embodiments, each unit dose metered from the dosing device does not vary by more than 5% at a temperature range from about 0° C. to less than 100° C., more preferably from about 10° C. to about 80° C., most preferably from about 20° C. to about 40° C. including room temperature. In yet another aspect of the invention, the dosing device is capable of metering each unit dose from the device such that it does not vary by more than 1% at a temperature range from about 0° C. to less than 100° C., more preferably from about 10° C. to about 80° C., most preferably from about 20° C. to about 40° C., including room temperature.

In most preferred embodiments, the dosing device of the invention provides a direct contact with the skin of a mammal. Such direct contact can be accomplished by providing the dosing device with an applicator having a surface that can easily come in direct contact with the skin of the mammal. Applicators that can accomplish this goal include those having a surface that can be either flat or convex, smooth or ridged. Moreover, when the device is held upright, the flat surface is preferably angled from a base line perpendicular to the dosing device. For example, a convex applicator surface can be provided by a roller ball placed in the applicator such that the unit dose can be rolled on the skin. In another aspect of the invention, the applicator can include a static surface rigidly connected with the housing and adapted to spread the dose onto the skin.

In certain embodiments, the applicator is preferably made from the material which inhibits microbial proliferation and/or is a non-wetting material which promotes the formation of droplets when exposed to moisture. In certain preferred embodiments, both the housing and the applicator of the dosing device are preferably comprised of a material which inhibits microbial proliferation, such as for example a silver containing plastic. Moreover, in certain other preferred embodiments, both the applicator and the housing of the dosing device are preferably comprised of a non-wetting material which promotes the formation of droplets when exposed to moisture, such as for example, silicone. The actuator can also be manufactured of the materials disclosed above

In other preferred embodiments, in order to prevent the decomposition of certain pharmaceutical formulations, the housing can be comprised of an aluminum lining or a plastic coated with aluminum. The applicator and/or the actuator can also be comprised of this material.

The dosing device of the present invention should contain the supply of pharmaceutical formulation with a tight seal from the external environment (e.g. from air, moisture and water) to provide many benefits. Such a configuration minimizes contamination of the contained formulation by contaminants and microbes. A tight seal also allows the device to be cleaned by a solvent, preferably water, without the formulation coming in contact with any of the liquid. Introduction of a liquid such as water into a semisolid formulation may hinder the accurate metering of the formulation if the device is adapted to deliver semisolids and the semisolid loses viscosity from the introduction of liquid. Preferably, the device of the present invention can be submerged in water for at least 30 seconds without consequence. Other embodiments can be submerged for a longer period, e.g., at least 2 hours, without consequence.

In certain embodiments of the invention, the applicator of the dosing device includes a valve disposed in an opening of the housing wherein upon actuation, the valve is movable between an open position to allow discharge of the unit dose through the opening to the applicator and a closed position to seal the opening. In certain aspects, the actuation of the actuator can cause a positive pressure in the housing of the device, which positive pressure can cause the valve to move from a closed position to an open position.

In certain other embodiments, the actuator comprises a button which upon actuation can cause the unit dose to be discharged from the housing to the applicator. Preferably, the button is positioned on the device to allow a user to actuate and then apply the unit dose to the skin with one hand. In preferred embodiments, the user does not have to reposition the hand from an actuation position to an application position and all steps of actuation and application can be performed with minimal or no repositioning of the hand.

In other embodiments, the actuator (preferably a button) is flush with the surface of the device or can be recessed. This minimizes the accidental actuation of an additional unit dose during the application process. In such embodiment, the actuator can be covered by the hand during application and will not discharge an undesired unit dose.

An actuator useful for the device of the invention can also comprise other types of mechanism for dispensing unit doses from the housing to the applicator. For example, the actuator can comprise a button, a rack, a pinion, and a lead screw in operative connection with each other, and the wherein actuation of the button causes the unit dose to be discharged from the housing to the applicator. Preferably, the dosing device further comprises a protective cover adapted to cover the valve and applicator in a closed position. A spring mechanism can also be used to move the valve from are open to a closed position.

In certain embodiments, the button can be moveable between a non-actuated position and an actuated position. When the actuation mechanism includes a lead screw, then the lead screw may preferably include a ratchet logic adapted to reduce the back pressure in the container. The lead screw may further comprise a valve logic for moving the valve from a closed position to an open position.

To avoid delivering a partial dose and/or contaminating the device, preferably, the dosing device of the invention further comprises a non-return mechanism adapted to prevent the actuator from delivering a partial dose and/or contaminating the device. Moreover, the actuator can also be adapted to substantially prevent air from entering the house during or after actuation.

In certain embodiments, the liquid contained in the device can be converted into a foam during the actuation process. Advantageously, the formulations to be included in the present invention can be formulated wherein the drug is substantially absorbed by the skin (e.g., 95% or more) over a period of less than about 30 minutes after administration, less than about 20 minutes after administration, or less than about 5 minutes after administration. In other embodiments, the dosing system of the invention provides for topical application of a pharmaceutical formulation wherein about 50% of the drug contained in the pharmaceutical formulation is absorbed by the skin over a period of more than about twelve (12) hours after hours after administration.

Therapeutic agents which can be used with the dosing system of the invention include all drugs which can be delivered on or through the skin for either a local or systemic effect. These compounds include drugs in all of the major therapeutic areas, including, but not limited to, ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, antipyretics and anti-inflammatory agents, androgens, local and general anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretic, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinson's agents, antiplatelet agents, antiprogestins, antithyroid agents, antitussives, antiviral agents, atypical antidepressants, azaspirodecanediones, barbituates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, 5-hydroxytryptamine antagonists, drugs for the treatment of hyperlipoproteinemia, hypnotics and sedatives, immunosupressive agents, laxatives, methylxanthines, monoamine oxidase inhibitors, neuromuscular blocking agents, organic nitrates, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and any therapeutically effective combinations thereof.

Representative drugs include, by way of example but not limited to, bepridil, diltiazem, felodipine, isradipine, nicardipine, nifedipine, nimodipine, nitredipine, verapamil, dobutamine, isoproterenol, carterolol, labetalol, levobunolol, nadolol, penbutolol, pindolol, propranolol, sotalol, timolol, acebutolol, atenolol, betaxolol, esmolol, metoprolol, albuterol, bitolterol, isoetharine, metaproterenol, pirbuterol, ritodrine, terbutaline, alclometasone, aldosterone, amcinonide, beclomethasone, dipropionate, betamethasone, clobetasol, clocortolone, cortisol, cortisone, corticosterone, desonide, desoximetasone, 11-desoxycorticosterone, 11-desoxycortisol, dexamethasone, diflorasone, fludrocortisone, flunisolide, fluocinolone, fluocinonide, fluorometholone, flurandrenolide, halcinonide, hydrocortisone, medrysone, 6.alpha.-methylprednisolone, mometasone, paramethasone, prednisolone, prednisone, tetrahydrocortisol, triamcinolone, benoxinate, benzocaine, bupivacaine, chloroprocaine, cocaine, dibucaine, dyclonine, etidocaine, lidocaine, mepivacaine, pramoxine, prilocalne, procaine, proparacaine, tetracaine, alfentanil, choroform, clonidine, cyclopropane, desflurane, diethyl ether, droperidol, enflurane, etomidate, halothane, isoflurane, ketamine hydrochloride, meperidine, methohexital, methoxyflurane, morphine, propofol, sevoflurane, thiamylal, thiopental, acetaminophen, allopurinol, apazone, aspirin, auranofin, aurothioglucose, colchicine, diclofenac, diflunisal, etodolac, fenoprofen, flurbiprofen, gold sodium thiomalate, ibuprofen, indomethacin, ketoprofen, meclofenamate, mefenamic acid, meselamine, methyl salicylate, nabumetone, naproxen, oxyphenbutazone, phenacetin, phenylbutazone, piroxicam, salicylamide, salicylate, salicylic acid, salsalate, sulfasalazine, sulindac, tolmetin, acetophenazine, chlorpromazine, fluphenazine, mesoridazine, perphenazine, thioridazine, trifluorperazine, triflupromazine, disopyramide, encainide, flecainide, indecainide, mexiletine, moricizine, phenyloin, procainamide, propafenone, quinidine, tocainide, cisapride, domperidone, dronabinol, haloperidol, metoclopramide, nabilone, prochlorperazine, promethazine, thiethylperazine, trimethobenzamide, buprenorphine, butorphanol, dezocine, diphenoxylate, drocode, hydrocodone, hydromorphone, levallorphan, levorphanol, loperamide, meptazinol, methadone, nalbuphine, nalmefene, nalorphine, naloxone, naltrexone, oxybutynin, pentazocine, isosorbide dinitrate, nitroglycerin, theophylline, phenylephrine, ephidrine, pilocarpine, furosemide, tetracycline, chlorpheniramine, ketorolac, bromocriptine, guanabenz, prazosin, doxazosin, flufenamic acid, pharmaceutically acceptable salts thereof and any therapeuctically effective combinations.

Other representative drugs useful with the dosing system the invention include without limitation, benzodiazepines, such as alprazolam, brotizolam, chlordiazepoxide, clobazam, clonazepam, clorazepate, demoxepam, diazepam, flumazenil, flurazepam, halazepam, lorazepam, midazolam, nitrazepam, nordazepam, oxazepam, prazepam, quazepam, temazepam, triazolam, and the like; an antimuscarinic agent such as anisotropine, atropine, clidinium, cyclopentolate, dicyclomine, flavoxate, glycopyrrolate, hexocyclium, homatropine, ipratropium, isopropamide, mepenzolate, methantheline, oxyphencyclimine, pirenzepine, propantheline, scopolamine, telenzepine, tridihexethyl, tropicamide, and the like; an estrogen such as chlorotrianisene, siethylstilbestrol, methyl estradiol, estrone, estrone sodium sulfate, estropipate, mestranol, quinestrol, sodium equilin sulfate, 17.beta.-estradiol (or estradiol), semi-synthetic estrogen derivatives such as the esters of natural estrogen, such as estradiol-17.beta.-enanthate, estradiol-17.beta.-valerate, estradiol-3-benzoate, estradiol-17.beta.-undecenoate, estradiol 16,17-hemisuccinate or estradiol-17.beta.-cypionate, and the 17-alkylated estrogens, such as ethinyl estradiol, ethinyl estradiol-3-isopropylsulphonate, and the like; an androgen such as danazol, fluoxymesterone, methandrostenolone, methyltestosterone, nandrolone decanoate, nandrolone phenpropionate, oxandrolone, oxymetholone, stanozolol, testolactone, testosterone, testosterone cypionate, testosterone enanthate, testosterone propionate, and the like; or a progestin such as ethynodiol diacetate, gestodene, hydroxyprogesterone caproate, levonorgestrel, medroxyprogesterone acetate, megestrol acetate, norethindrone, norethindrone acetate, norethynodrel, norgestrel, progesterone, pharmaceutically acceptable salts thereof and any therapeutically effective combinations thereof.

Therapeutic agents having local activity which can be used with the dosing system of the invention include, for example, active substances for use in the treatment of disorders of the skin, such disorders including, by way of example, psoriasis, eczema, acne, nappy rash, other inflammatory disorders, bacterial infections, viral infections, fungal infections, anaphylactic conditions, malignancies and warts.

Advantageously, therapeutic agents having local activity for use with the dosing system of the invention can be selected from the group consisting of local anesthetics, corticosteroids, antibacterial agents; antifungal agents or any therapeutically effective combination thereof.

More particularly, therapeutic agents having local activity for use with the system of the invention can be selected from the group consisting of tetracaine, benzocaine, lindocaine, hydrocortisone, beclomethasone diproprionate, clobetasol proprionate, fluticasone proprionate, ichthaminol, lithium succinate, coal tar, dithranol, benzoyl peroxide, tretinoin, sulphur, vitamin D and derivatives thereof, framycetin, chlortetracycline hydrochloride, fusidic acid, clotrimazole, econazole, amorolfine and terbenafine, or any therapeutically effective combination thereof.

Local anesthetics include without limitation an anesthetic selected from the group consisting of bupivacaine, levo-bupivacaine, ropivacaine, benzocaine, dibucaine, procaine, chloroprocaine, prilocaine, mepivacaine, etidocaine, tetracaine, lidocaine, and xylocaine, as well as anesthetically active derivatives, analogs, isomers and mixtures thereof.

In embodiments where the therapeutic drugs produce a local effect, active agents include without limitation antiviral agents (e.g., acyclovir and idoxuridine, etc.), antifungal agents (e.g., amphotericin B, clotrimazole, nystatin, ketoconazole, miconazole, butocouazole, haloprogin, etc.), antibiotic agents (penicillins, cephalosporins erythromycin, tetracycline, clindamycin, aminoglycosides, chloramphenicol, polymixin b, bacitracin, neomycin, gentamycin etc.), antiseptics (e.g., povidone-iodine, methylbenzethonium chloride, etc.), antiparasitics (e.g., lindane, anthralin, etc.) analgesic agents (e.g., methylsalicylate, salicylic acid, dyclonine, aloe vera etc.), local anesthetics (e.g., benzocaine, lidocaine, xylocalne, butamben picrate, etc.), anti-inflammatory agents (e.g., steroidal compounds such as dexamethasone, betamethasone, prednisone, prednisolone, triamcinolone, hydrocortisone, alclometasone, amcinonide, diflorasone, etc. as well as non-steroidal anti-inflammatories), anti-itch and irritation-reducing compounds (e.g., antihistamines such as diphenhydramine and psoriasis treatments); burn relief compounds (e.g., o-amino-p-toluenesulfonamide, monoacetate, etc.); depigmenting agents (e.g., monobenzone); and hormonal agents (e.g., oestriol).

In certain embodiments of the invention, the drug included in the pharmaceutical formulation comprises a pharmaceutically acceptable source of nitrites. In certain embodiments, the nitrites are included with a pharmaceutically acceptable acidifying agent as disclosed in WO 95/22335. In other embodiments, the drug can include a composition comprising an aqueous solution of nitric acid and nitrous acid as disclosed in U.S. Pat. No. 4,595,591. In other embodiments, the composition can comprise a vaso-active composition comprising nitrogen oxide generated from an admixture of ferrous sulphate, an organic acid and an inorganic nitrite as disclosed in U.S. Pat. No. 5,648,101. In other embodiments the nitrogen oxide can be used to inhibit viruses as disclosed in WO 96/02268. In other embodiments, the composition can comprise nitrous oxide in combination with a fatty acid or a lower alkyl ester thereof as disclosed in WO 93/25213. The disclosure of all of these references are incorporated by reference in their entireties for all purposes.

In other embodiments comprising nitrogen oxides, the nitrogen oxide is produced when a pharmaceutically acceptable acidifying agent and a pharmaceutically acceptable donor of nitrogen oxides or precursor thereof are brought into contact at the site of action as disclosed in WO 99/44622 hereby incorporated by reference in its entirety for all purposes.

In certain embodiments of the invention, the device can comprise two housings, each containing a separate drug. This would be useful where it is desirable to have two formulations dispensed simultaneously as in WO 99/44622. The two formulations can both be dispensed upon actuation where they mix upon movement to the applicator. In other embodiments, the formulations can be actuated separately and then administered to the patient sequentially and mixed upon application of the second formulation.

In other embodiments, the formulation can contain a permeation enhancer which is known in the art to improve the absorption of the drug. Such permeation enhancers are disclosed in WO 99/24036 hereby incorporated by reference in its entirety for all purposes.

In other embodiments intended for local delivery, the pharmaceutical composition can comprise zinc ions which improve efficacy by enhancing skin penetration and reduce the risk of side effects by discouraging the passage of the drugs through the skin to the underlying systemic circulation.

Pharmaceutical formulations which are useful with the dosing system of the invention include all pharmaceutically acceptable salts and conjugates thereof. Other topically-active compounds are listed in Remington's Pharmaceutical Sciences, 17th Ed., Merck Publishing Co., Easton, Pa. (1985), pages 773-791 and pages 1054-1058 (hereinafter Remington's), incorporated herein by reference.

The dosing system of the present invention can also be used for topical application of other preparations, such as for cosmetic purposes, e.g., antiperspirants, sunblocks, keratolitics, skin softeners, fragrances and anti-acne preparations.

These agents include sun screens such as p-dimethylaminobenzoic acid; skin softeners such as urea; keratolytic agents such as salicylic acid; acne agents such as benzoyl peroxide, perfumes and the like.

Suitable antiperspirant compositions include astringent salts. The astringent salts include organic and inorganic salts of aluminum, zirconium, zinc, and mixtures thereof. The anion of the astringent salt can be, for example, sulfate, chloride, chlorohydroxide, alum, formate, lactate, benzyl sulfonate or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides. Exemplary zirconium compounds include zirconium oxy salts and zirconium hydroxy salts, also referred to as zirconyl salts and zirconyl hydroxy salts.

Exemplary antiperspirant compounds therefore include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohydrex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichlorohydrex PG, aluminum chloride, aluminum zirconium pentachlorohydrate, and mixtures thereof. Numerous other useful antiperspirant compounds are listed in WO 91/19222 and in the Cosmetic and Toiletry Fragrance Handbook, The Cosmetic, Toiletry and Fragrance Association, Inc., Washington, D.C., p. 56, 1989, hereinafter the CTFA Handbook, incorporated herein by reference.

Therapeutic agents having non-local activity for use with the dosing system of the invention include without limitation, active substances for use in the treatment or prevention of various systemic and disorders and their symptoms, such as disorders of the cardiovascular system, disorders of the muscles or joints, disorders of the organs. More particularly, therapeutic agents having non-local activity for use according to any aspect of the present invention include, for example, active substances for use as vasodilators, active substances for the treatment of motion sickness, contraceptive agents, hormone replacement agents, painkillers, smoking cessation aids, or any therapeutically effective combination thereof.

Advantageously, therapeutic agents having non-local activity for use according to any aspect of the present invention are selected from the group consisting of nitroglycerin, scopolamine, estradiol, norethisterone, fentanyl and nicotine, or any therapeutically effective combination thereof.

Pharmaceutical formulations useful with the dosing system of the present invention may include any suitable carrier for topical delivery. Suitable carriers include polymers such as sodium alginate, gelatin, corn starch, gum tragacanth, methylcellulose, hydroxyethylcellulose, carboxymethylcellulose, xanthan gum, dextrin, carboxymethylstarch, polyvinyl alcohol, sodium polyacrylate, methoxyethylene-maleic anhydride copolymer, polyvinyl ether, polyvinylpyrrolidone.

The carrier can be a cellulose, one or more glycerides (such as for example, one or more glycerol esters of saturated acids or one or more polyglycolysed glycerides, cocoa butter, theobroma or the like), one or more high molecular weight polyethylene glycol, one or more polyoxyethylene, lanolin and derivatives thereof, and one or more fatty acids, fatty alcohols, fatty acid esters (including, for example, caprylic acid, caprylic triglyceride or the like), any of which preceding ingredients can be optionally mixed with one or more organic oils (including, for example hydrogenated vegetable oils) or the like.

A carrier medium suitable for use in a pharmaceutical formulation useful with the dosing system of the invention can comprise a wax, a fat an oil or a combination thereof, including, without limitation, for example beeswax, olive oil, cocoa butter, sesame oil, soybean oil, camellia oil, peanut oil, beef fat, lard and lanolin.

In certain embodiments of the carrier medium comprises white petrolatum or a paraffin. In other embodiments the carrier medium includes a higher fatty acid, for example stearic acid. In certain embodiments the carrier comprises a higher alcohol, such as for example, cetyl alcohol, stearyl alcohol and combinations thereof. In other aspects of the invention, the carrier comprises the polyethylene glycol or water.

Any and all combinations of pharmaceutical excipients which provide a suitable vehicle for the drug when used in the present invention are meant to be encompassed by the present invention. Further excipients are know to those skilled in the art as described in Remington's Pharmaceutical Sciences, 17th Ed., Merck Publishing Co., Easton, Pa. (1985), incorporated by reference.

The present invention also relates to method for topically administering a pharmaceutical formulation to a skin of a mammal, the method including: (i) actuating a dosing system comprising a dosing device including: a housing holding at least one unit dose of a pharmaceutical formulation comprising a drug and a carrier medium therefore; an applicator adapted for topically administering at least a unit dose of the pharmaceutical formulation directly onto the skin of the mammal; and an actuator, wherein upon actuation, the dosing device meters a unit dose of the pharmaceutical formulation from the housing to the applicator; and (ii) applying the unit dose directly onto the skin of the mammal with the applicator.

In certain preferred embodiments, the method for topically administering a pharmaceutical formulation further includes re-actuating the dosing system and administering additional unit doses of the pharmaceutical formulation. The method of the present invention is applicable for topically administering pharmaceutical formulations wherein the drug provides a local or a systemic effect.

In another aspect, the present invention relates to, in part, a method of preparing the dosing system for topical delivery of a pharmaceutical formulation including preparing at least one unit dose of a pharmaceutical preparation comprising a drug and a carrier; and placing the at least one unit dose into a dosing device comprising
a housing for holding the at least one unit dose of a pharmaceutical; an applicator adapted for topically administering a unit dose of the pharmaceutical formulation directly onto the skin; and an actuator, wherein upon actuation, the device meters a unit dose of the pharmaceutical formulation from the housing to the applicator.

### DETAILED DESCRIPTION OF CERTAIN EMBODIMENTS

FIG. 1 shows the exterior one embodiment of dosing device 10 with protective cap 11 shown in a closed position attached to an upper end of main body 12. Protective cap 11 may include side portions 14 to provide an attachment to main body 12 also in an opened position, which will be explained further below. Main body 12 may have a generally cylindrical shape and device 10 may be sized so as to be hand-held. Button 13 may be disposed at a lower end of main body 12.

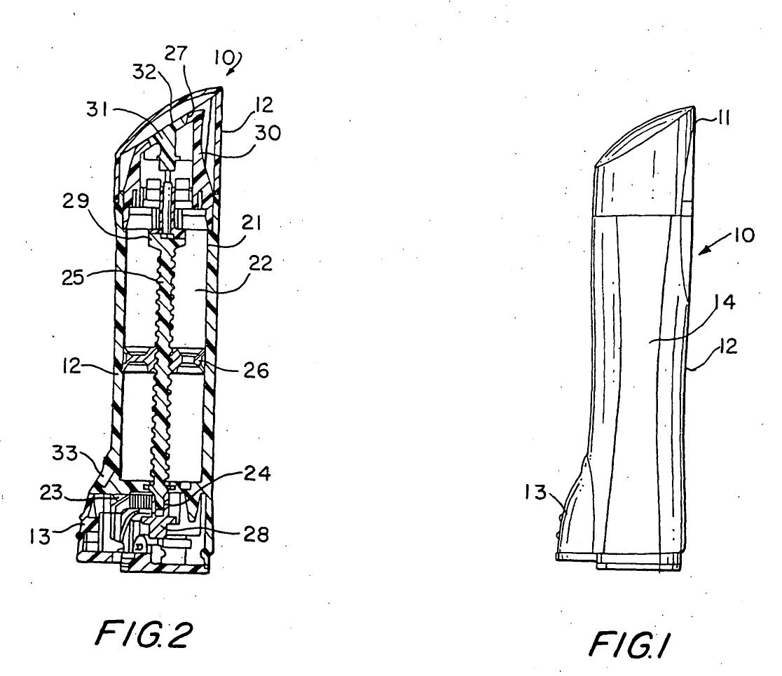

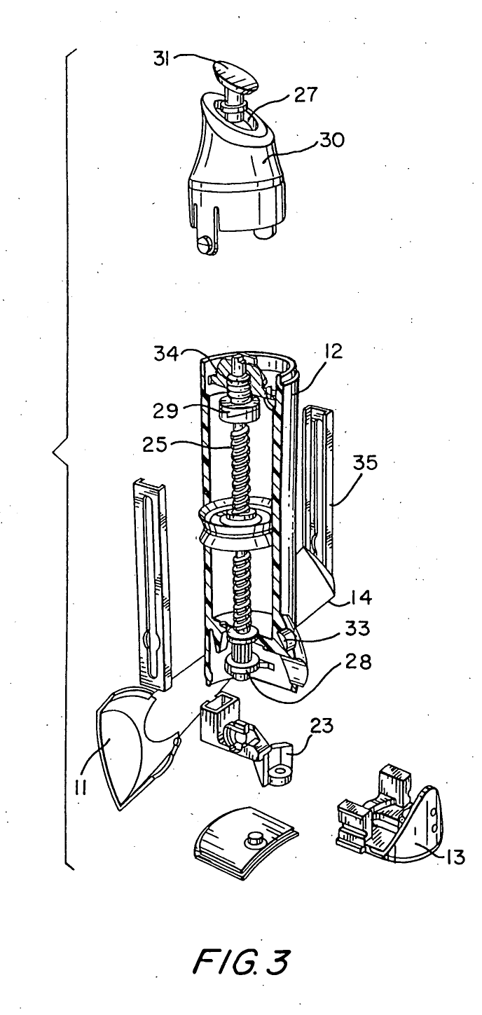

Referring to FIGS. 2 and 3, pharmaceutical formulation 22, which may include a drug and a carrier, is held within cylindrical housing 21 of main body 12. Button 13 may be rigidly connected to rack 23, which is in operative connection with pinion 24, so that a displacement of rack 23 in its longitudinal direction causes pinion 24 to rotate. Pinion 24 may be rigidly connected to lead screw 25 which may be disposed longitudinally within housing 21. Lead screw 25 in turn may be operatively connected to piston 26 in such a way so that a rotation of lead screw 25 causes a displacement of piston 26 in a longitudinal direction of lead screw 25. Piston 26 may be disc-shaped and extend from lead screw 25 at its center to an inner wall of housing 21 at its outer perimeter. Preferably a seal is formed between piston 26 and both lead screw 25 and housing 25, so that upon actuation of button 13, rack 23 causes pinion 24 and lead screw 25 to rotate, thus causing piston 26 to move incrementally in an upward direction pushing pharmaceutical formulation 22 upward with it. Button 13, rack 23, pinion 24, lead screw 25, piston 26 and housing 21 may be configured, for example, so that each time button 13 is fully depressed, lead screw 25 rotates 120 degrees, thus causing piston 26 to move upward 1 mm along lead screw 25 so as to discharge 0.5 grams of pharmaceutical compound 22 through outlet opening 27 of housing 21 and onto applicator head 30. Outlet valve 31 covers outlet opening 27 in its closed position. Outer surface 32 of outlet valve 31 forms part of applicator head in that it is used to apply pharmaceutical formulation 22 to the skin (not shown). Each successive actuation of button 13 causes a metered unit dose (for example 0.5 grams) of pharmaceutical formulation 22 to discharge from outlet opening 27 and onto applicator head 30. Dose counter window 33 may be disposed in main body 12 of device 10 to enable a user to read a display of a counter (not shown) in order to know how many unit doses of pharmaceutical formulation 22 have been discharged.

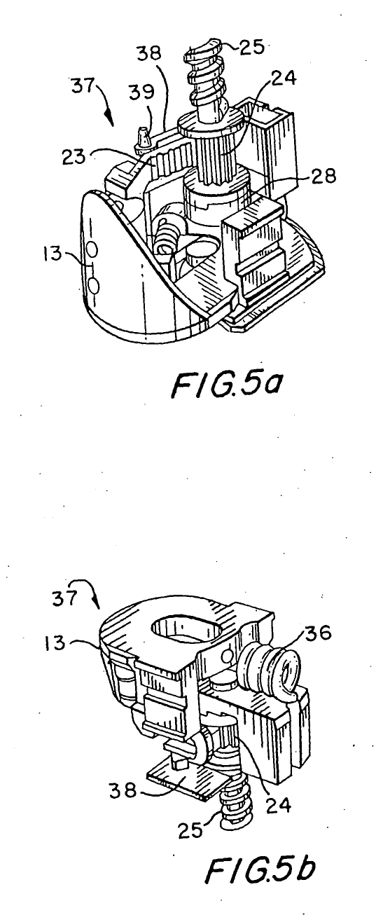

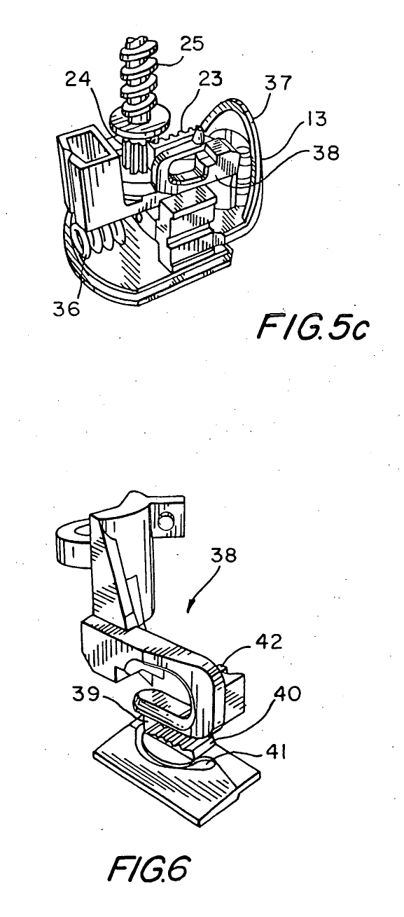

Lead screw 25, at its lower end, may be operatively connected to lower logic 28. Lower logic 28 may be configured to cause lead screw to displace in a longitudinally upward direction (for example by 1 mm) as button 23 is depressed, and to drop back down again to its original longitudinal position when button 23 reaches its fully depressed position or when button 23 travels back to its original (not actuated) position, (see also FIG. 5a). This action serves to prevent backpressure from building up within housing 21, which might otherwise cause device 10 to leak.

FIG. 3 shows a section view of protective cover 11 in an open position, i.e. not attached to the upper end of main body 12. Protective cover 11 may include side portions 14 in operative connection with side clamps 35—for example via pins of side portions 14 (not shown) and slots in side clamps 35—to provide a connection to main body 12 in both closed and opened positions. This feature serves to prevent protective cover 11 from being misplaced during use. Other features such as a tether, hinge, or other connection between protective cover 11 and main body 12 may be used instead to provide a similar function. In its closed position, protective cover 11 serves to protect applicator head 30 from contamination. Side portions 14 and side clamps 14 may be configured to enable protective cap 11 to attach to the lower end of main body 12 so as not to interfere with use of dosing device 10.

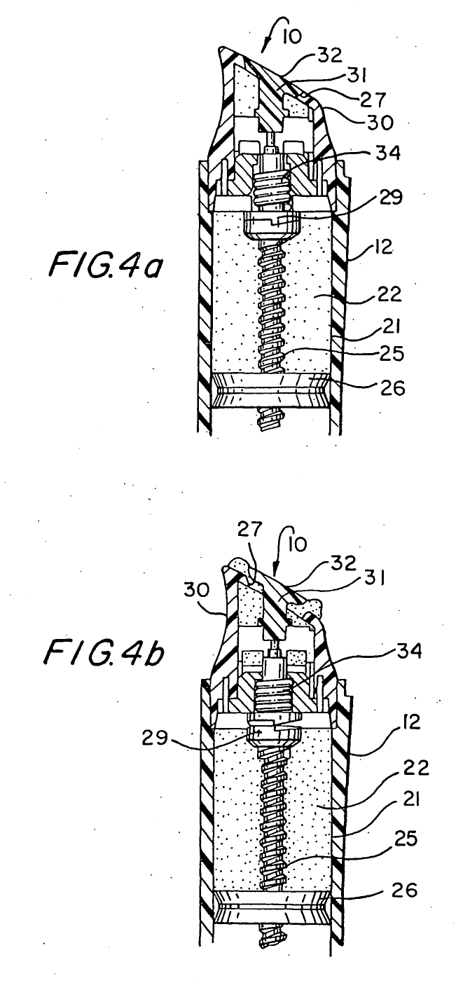

Lead screw 25, at its lower end, may be operatively connected with upper logic 29, which may include, for example, a pair of disk-shaped components having opposing ramps. In the example in which a complete depression of button 13 causes a 120 degree rotation of lead screw 25, the disk shape components may be configured to have three opposing ramps, each covering a 120 degree arc of the disk. FIG. 4a shows an upper portion of device 10 with outlet valve in a closed position and FIG. 4a shows the upper portion of device 10 with outlet valve 31 in an opened position. When lead screw 25 rotates, for example upon actuation of button 13, upper logic 29 may function to lift outlet valve 31 so that outlet valve 31 reaches its maximum height at the end of the incremental rotation movement of lead screw 25 (i.e. when button 13 is in its fully depressed position). For example, if lower logic 28 is configured to displace lead screw 25 in a longitudinal direction of 1 mm during a single actuation of button 13, then upper logic 29 may be configured to displace outlet valve 31 in an upward direction by approximately 2 mm, in order to provide ample room in outlet opening 27 for discharge of pharmaceutical formulation 22. Outlet valve spring 34 may be disposed between outlet valve 31 and housing 21 to provide tension between the two components tending to move outlet valve 31 to a closed position. Thus, when lead screw 25 reaches the end of its incremental rotational movement, upper logic 29 and outlet valve spring 34 cause outlet valve 31 to move to its closed position. During the time that the valve is opened a metered unit dose of pharmaceutical formulation is discharged through outlet opening 27 and onto applicator head 30.

FIGS. 5a, 5b, and 5c show perspective views of actuator 37 according to the present invention. As described above, actuation of button 13 may cause displacement of rack 23 to rotate pinion 24, thus rotating lead screw 25. Lower logic 28, comprising in this embodiment two opposing ramped disks cause lead screw 25 to displace in a longitudinally upward direction during one incremental rotational movement, and to drop back down to its original longitudinal position when button 13 is fully depressed or when button 13 returns to its non-depressed position. Button return spring 36 is disposed between button 13 and main body 22 so as to tend to move button 13 to its non-depressed position.

FIG. 6 shows a perspective view of one embodiment of a non-return mechanism that may be used with the dosing device 10 of FIG. 1. In a dosing device according to the present invention it is desirable to prevent a user from dispensing an amount of pharmaceutical formulation other than the predetermined metered unit dose. Therefore, it may be desirable to incorporate a non-return mechanism such as, for example, non-return mechanism 38 of FIG. 6, in order to prevent a user from partially actuating the actuator and thus dispensing less than the full unit dose upon actuation of the device.

Referring to FIG. 6, non-return mechanism 38 includes pin 39 in operative connection with ramp platform 40. Pin 39 may be rigidly connected to button 13 (not shown in FIG. 6) and ramp platform 40 may be rigidly connected to main body 12 (not shown in FIG. 6). Likewise, pin 39 may be rigidly connected to main body 12 with ramp rack 40 rigidly connected to button 13. Pin compression spring 42 provides a force tending to keep pin 39 in contact with ramp platform 40. As button 13 is depressed pin 39 moves along ramp platform 40, and is able to ride up over reach of the ramps on ramp platform 40 in one direction, but is prevented by a ratchet effect from traveling over the ramps in the opposite direction. Thus, if button 13 is not fully depressed, anti-return mechanism 38 prevents button 13 from returning to its non-depressed position because pin 39 can only travel in one direction along ramp rack 40. Ramp rack 40 may have a length corresponding to a travel distance of button 13 so that when button 13 reaches its fully depressed position, pin 39 reaches the end of ramp rack 40 and falls into return track 41. Track 41 guides pin 39 around ramp rack 40 as button 13 travels back to its non-depressed position (via force provided by button return spring 36 (not shown in FIG. 6) so that pin 39 is returned to its starting position at the beginning end of ramp rack 40.

The present invention has been described herein with reference to specific exemplary embodiments thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense. A person of skill in the art will, of course, appreciate many ways of implementing the present invention, in addition to the embodiments described in FIGS. 1-6. For example, many well-known structures can be used instead of using a piston and lead screw to create pressure on the pharmaceutical formulation within the housing. For instance, if the housing were a flexible tube, a roller or pair of rollers could be used to squeeze the tube and cause the formulation to be discharged. The push may be replaced by a sliding button, lever, or rotary knob. An actuator could control movement of the rollers to cause a metered unit dose of the formulation to be discharged. The formulation within the housing may also, for example, be pre-pressurized using a propellant and the actuator could cause metered unit dosages to be discharged using by actuating a metering valve.

1. A dosing device for topically administering a pharmaceutical formulation to the skin of a mammal, the device comprising:
a housing capable of storing at least one unit dose of a pharmaceutical formulation comprising a drug incorporated with a pharmaceutically acceptable carrier suitable for topical application onto the skin of said mammal;
an applicator adapted for topically administering a unit dose of the pharmaceutical formulation directly onto the skin; and
an actuator capable of metering a single unit dose of the pharmaceutical formulation from a first position in which the unit dose is stored in the housing to a second position in which the single unit dose is external to the device on the applicator so that the single unit dose can be topically administered.

2. The dosing device of claim 1, wherein said housing is capable of holding multiple unit doses of the pharmaceutical formulation.

3. The dosing device of claim 1, wherein said housing is adapted to hold a semisolid pharmaceutical formulation.

4. The dosing device of claim 3, wherein said housing is adapted to hold a semisolid pharmaceutical formulation selected from the group consisting of an ointment, gel, emulsion, lotion, spray, cream and paste.

5. The dosing device of claim 1, wherein said housing is adapted to hold a liquid pharmaceutical formulation.

6. The dosing device of claim 5, wherein said housing is adapted to hold a liquid pharmaceutical formulation selected from the group consisting of a suspension and a solution.

7. The dosing device of claim 1 wherein upon actuation, the device is capable of metering a unit dose of pharmaceutical formulation from about 0.10 grams to about 5 grams from the housing to the applicator.

8. The dosing device of claim 7 wherein upon actuation, the device is capable of metering a unit dose of pharmaceutical formulation of about 1.0 gram from the housing to the applicator.

9. The dosing device of claim 1 wherein upon a subsequent actuation, an additional unit dose is capable of being delivered from the housing to the applicator, until depletion of the pharmaceutical formulation from the housing.

10. The dosing device of claim 9 further comprising a means for preventing the actuator from functioning after a predetermined number of actuations, for a predetermined time period.

11. The dosing device of claim 10 wherein the desired time period is the dosing interval of the drug.

12. The dosing device of claim 11, wherein the dosing interval is from about 1 hour to about 24 hours.

13. The dosing device of claim 11, wherein the dosing interval is from about 4 hour to about 12 hours.

14. The dosing device of claim 10, wherein the predetermined number of actuation is a number which administers the prescribed amount of pharmaceutical formulation.

15. The dosing device of claim 14, wherein the predetermined number of actuations is one actuation.

16. The dosing device of claim 14, wherein the predetermined number of actuations is more than one actuation.

17. The dosing device of claim 10 wherein the desired time period is at least the time needed to topically administer the previously metered unit dose, in order to prevent accidental actuation during application.

18. The dosing device of claim 1 wherein each unit dose metered from the device does not vary by more than about 10% at room temperature.

19. The dosing device of claim 1 wherein each unit dose metered from the device does not vary by more than about 10% at a temperature from about 20° C. to about 40° C.

20. The dosing device of claim 1 wherein each unit dose metered from the device does not vary by more than about 10% at a temperature from about 10° C. to about 80° C.

21. The dosing device of claim 1 wherein each unit dose metered from the device does not vary by more than about 10% at a temperature from greater than 0° C. to less than about 100° C.

22. The dosing device of claim 1 wherein each unit dose metered from the device does not vary by more than about 5% at room temperature.

23. The dosing device of claim 1 wherein each unit dose metered from the device does not vary by more than about 5% at a temperature from about 20° C. to about 40° C.

24. The dosing device of claim 1 wherein each unit dose metered from the device does not vary by more than about 5% at a temperature from about 10° C. to about 80° C.

25. The dosing device of claim 1 wherein each unit dose metered from the device does not vary by more than about 5% at a temperature from greater than 0° C. to less than about 1000 C.

26. The dosing device of claim 1 wherein each unit dose metered from the device does not vary by more than about 1% at room temperature.

27. The dosing device of claim 1 wherein each unit dose metered from the device does not vary by more than about 1% at a temperature from about 20° C. to about 40° C.

28. The dosing device of claim 1 wherein each unit dose metered from the device does not vary by more than about 1% at a temperature from about 10° C. to about 800 C.

29. The dosing device of claim 1 wherein each unit dose metered from the device does not vary by more than about 1% at a temperature from greater than 0° C. to less than 1000 C.

30. The dosing device of claim 1 wherein said applicator comprises a flat surface.

31. The dosing device of claim 30 wherein said flat surface is angled from a baseline perpendicular to said device when said device is held upright.

32. The dosing device of claim 1 wherein said applicator comprises a convex surface.

33. The dosing device of claim 1 wherein said applicator is comprised of a material which inhibits microbial proliferation.

34. The dosing device of claim 1 wherein said housing is comprised of a material which inhibits microbial proliferation.

35. The dosing device of claim 33 wherein said material comprises a silver containing plastic.

36. The dosing device of claim 1 wherein said applicator is comprised of a non-wetting material which promotes the formation of droplets when exposed to moisture.

37. The dosing device of claim 1 wherein said housing is comprised of a non-wetting material which promotes the formation of droplets when exposed to moisture.

38. The dosing device of claim 36 wherein said material comprises silicone.

39. The device of claim 30 wherein said applicator surface is smooth.

40. The device of claim 30 wherein said applicator surface is ridged.

41. The dosing device of claim 1 further comprising a counter which indicates the number of unit doses remaining in the system.

42. The dosing device of claim 1 further comprising a counter which indicates the number of doses actuated.

43. The device of claim 1 wherein after depletion or partial depletion of said at least one unit dose, said device is capable of being reloaded with at least one additional unit dose.

44. The device of claim 1 wherein after depletion of said at least one unit dose, said device is incapable of being reloaded and is disposable.

45. The device of claim 2 wherein said housing is adapted to contain at least 5 unit doses of said pharmaceutical formulation.

46. The device of claim 2 wherein said housing is adapted to contain from about 5 doses to about 400 unit doses of said pharmaceutical formulation.

47. The device of claim 2 wherein said housing is adapted to contain from about 40 doses to about 120 unit doses of said pharmaceutical formulation.

48. The device of claim 2 wherein said housing is adapted to contain from about 30 doses to about 100 unit doses of said pharmaceutical formulation.

49. The device of claim 2 wherein said housing is adapted to contain about 365 unit doses of said pharmaceutical formulation.

50. The device of claim 2 wherein said housing is adapted to contain about 30 unit doses of said pharmaceutical formulation.

51. The dosing device of claim 1 wherein the applicator includes a valve disposed in an opening of the housing wherein upon actuation, the valve is moveable between an open position to allow discharge of the unit dose through the opening to the applicator and a closed position to seal the opening.

52. The dosing device as recited in claim 51 wherein actuation of the actuator causes a positive pressure in the housing, the positive pressure causing the valve to move from the closed position to the open position.

53. The dosing device of claim 1 wherein said actuator comprises a button wherein actuation of the button causes a unit dose to be discharged from the housing to the applicator.

54. The dosing device as recited in claim 1 wherein the actuator comprises a button, a rack, a pinion, and a lead screw in operative connection with each other, and wherein actuation of the button causes the unit dose to be discharged from the housing to the applicator.

55. The dosing device as recited in claim 51 further comprising a protective cover adapted to cover the valve and applicator in a closed position.

56. The dosing device as recited in claim 51 wherein said valve is movable from the open position to the closed position by a spring mechanism.

57. The dosing device as recited in claim 53 wherein the button is moveable between a non-actuated position and an actuated position.

58. The dosing device as recited in claim 57 further comprising a button spring mechanism which causes the button to return to the non-actuated position after actuation.

59. The dosing device as recited in claim 1 further comprising a non-return mechanism adapted to prevent the actuator from delivering a partial dose and/or contaminating the device.

60. The dosing device as recited in claim 54 wherein the lead screw includes a ratchet logic adapted to reduce a back pressure in the container.

61. The dosing device as recited in claim 54 wherein the lead screw further comprises a valve logic for moving the valve from the closed position to the open position.

62. The dosing device as recited in claim 1 wherein air is substantially prevented from entering the housing during or after actuation.

63. The dosing device as recited in claim 10 wherein said means is a mechanical means or an electrical means.

64. The dosing device as recited in claim 1 wherein the applicator includes a roller ball adapted to roll the unit dose onto the skin.

65. The dosing device as recited in claim 1 wherein the applicator comprises a static surface rigidly connected with the housing and adapted to spread the dose onto the skin.

66. A dosing system comprising a pharmaceutical formulation comprising a drug and a carrier suitable for topical application contained in a device of claims 1-65.

67. The system of claim 66, wherein said pharmaceutical formulation is a semisolid

68. The system of claim 67, wherein said semisolid is selected from the group consisting of an ointment, gel, emulsion, lotion, spray, cream and paste.

69. The system of claim 66, wherein said pharmaceutical formulation is a liquid.

70. The system of claim 69 wherein said liquid is a suspension or a solution.

71. The system of claim 69 wherein said liquid is converted to a foam upon actuation and metering of a unit dose from said housing to said applicator.

72. The system of claim 66, wherein at least about 95% of said drug is absorbed by the skin over a period of less than about 30 minutes after administration.

73. The system of claim 72, wherein at least about 95% of said drug is absorbed by the skin over a period of less than about 20 minutes after administration.

74. The system of claim 72, wherein at least about 95% of said drug is absorbed by the skin over a period of less than about 5 minutes after administration.

75. The system of claim 66, wherein not more than about 50% of said drug is absorbed by the skin over a period of about 12 hours or more after administration.

76. The system of claim 75, wherein not more than about 50% of said drug is absorbed by the skin over a period of about 6 hours or more after administration.

77. The system of claim 75, wherein not more than about 50% of said drug is absorbed by the skin over a period of about 2 hours or more after administration.

78. The system of claim 66 wherein said drug is selected from the group consisting of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha₂-adrenergic agonists, analgesics, antipyretics, anti-inflammatory agents, androgens, local anesthetics, general anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretic agents, antiemetic agents, prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinson agents, antiplatelet agents, antiprogestins, antithyroid agents, antitussives, antiviral agents, antidepressants, azaspirodecanediones, barbituates, benzodiazepines, benzothiadiazides, beta-adrenergic agonists, beta-adrenergic antagonists, selective beta₁-adrenergic antagonists, selective beta₂-adrenergic agonists, bile salts, agents affecting volume and composition of body fluids, butyrophenones, agents affecting calcification, calcium channel blockers, cardiovascular drugs, catecholamines, sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity, agents for treatment of peptic ulcers, hematopoietic agents, anti-histamines, 5-hydroxytryptamine antagonists, drugs for the treatment of hyperlipoproteinemia, hypnotics, sedatives, immunosupressive agents, laxatives, bronchodilators, monoamine oxidase inhibitors, neuromuscular blocking agents, organic nitrates, pancreatic enzymes, phenothiazines, progestins, prostaglandins, anti-psychotic agents, retinoids, sodium channel blockers, agents for spasticity, agents for acute muscle spasms, succinimides, xanthines, thrombolytic agents, thyroid agents, tricyclic antidepressants, inhibitors of tubular transport of organic compounds, drugs affecting uterine motility, vasodilators and vitamins.

79. The system of claim 66 wherein said drug is selected from the group consisting of bepridil, diltiazem, felodipine, isradipine, nicardipine, nifedipine, nimodipine, nitredipine, verapamil, dobutamine, isoproterenol, carterolol, labetalol, levobunolol, nadolol, penbutolol, pindolol, propranolol, sotalol, timolol, acebutolol, atenolol, betaxolol, esmolol, metoprolol, albuterol, bitolterol, isoetharine, metaproterenol, pirbuterol, ritodrine, terbutaline, alclometasone, aldosterone, amcinonide, beclomethasone, dipropionate, betamethasone, clobetasol, clocortolone, cortisol, cortisone, corticosterone, desonide, desoximetasone, 11-desoxycorticosterone, 11-desoxycortisol, dexamethasone, diflorasone, fludrocortisone, flunisolide, fluocinolone, fluocinonide, fluorometholone, flurandrenolide, halcinonide, hydrocortisone, medrysone, 6.alpha.-methylprednisolone, mometasone, paramethasone, prednisolone, prednisone, tetrahydrocortisol, triamcinolone, benoxinate, benzocaine, bupivacaine, chloroprocaine, cocaine, dibucaine, dyclonine, etidocaine, lidocaine, mepivacaine, pramoxine, prilocalne, procaine, proparacaine, tetracaine, alfentanil, choroform, clonidine, cyclopropane, desflurane, diethyl ether, droperidol, enflurane, etomidate, halothane, isoflurane, ketamine hydrochloride, meperidine, methohexital, methoxyflurane, morphine, propofol, sevoflurane, thiamylal, thiopental, acetaminophen, allopurinol, apazone, aspirin, auranofin, aurothioglucose, colchicine, diclofenac, diflunisal, etodolac, fenoprofen, flurbiprofen, gold sodium thiomalate, ibuprofen, indomethacin, ketoprofen, meclofenamate, mefenamic acid, meselamine, methyl salicylate, nabumetone, naproxen, oxyphenbutazone, phenacetin, phenylbutazone, piroxicam, salicylamide, salicylate, salicylic acid, salsalate, sulfasalazine, sulindac, tolmetin, acetophenazine, chlorpromazine, fluphenazine, mesoridazine, perphenazine, thioridazine, trifluorperazine, triflupromazine, disopyramide, encainide, flecainide, indecainide, mexiletine, moricizine, phenyloin, procainamide, propafenone, quinidine, tocainide, cisapride, domperidone, dronabinol, haloperidol, metoclopramide, nabilone, prochlorperazine, promethazine, thiethylperazine, trimethobenzamide, buprenorphine, butorphanol, dezocine, diphenoxylate, drocode, hydrocodone, hydromorphone, levallorphan, levorphanol, loperamide, meptazinol, methadone, nalbuphine, nalmefene, nalorphine, naloxone, naltrexone, oxybutynin, pentazocine, isosorbide dinitrate, nitroglycerin, theophylline, phenylephrine, ephidrine, pilocarpine, furosemide, tetracycline, chlorpheniramine, ketorolac, bromocriptine, guanabenz, prazosin, doxazosin, flufenamic acid and pharmaceutically acceptable salts thereof.

80. The system of claim 66 wherein said drug is selected from the group consisting of benzodiazepines, such as alprazolam, brotizolam, chlordiazepoxide, clobazam, clonazepam, clorazepate, demoxepam, diazepam, flumazenil, flurazepam, halazepam, lorazepam, midazolam, nitrazepam, nordazepam, oxazepam, prazepam, quazepam, temazepam, triazolam, and the like; an antimuscarinic agent such as anisotropine, atropine, clidinium, cyclopentolate, dicyclomine, flavoxate, glycopyrrolate, hexocyclium, homatropine, ipratropium, isopropamide, mepenzolate, methantheline, oxyphencyclimine, pirenzepine, propantheline, scopolamine, telenzepine, tridihexethyl, tropicamide, and the like; an estrogen such as chlorotrianisene, siethylstilbestrol, methyl estradiol, estrone, estrone sodium sulfate, estropipate, mestranol, quinestrol, sodium equilin sulfate, 17.beta.-estradiol (or estradiol), semi-synthetic estrogen derivatives such as the esters of natural estrogen, such as estradiol-17.beta.-enanthate, estradiol-17.beta.-valerate, estradiol-3-benzoate, estradiol-17.beta.-undecenoate, estradiol 16,17-hemisuccinate or estradiol-17.beta.-cypionate, and the 17-alkylated estrogens, such as ethinyl estradiol, ethinyl estradiol-3-isopropylsulphonate, and the like; an androgen such as danazol, fluoxymesterone, methandrostenolone, methyltestosterone, nandrolone decanoate, nandrolone phenpropionate, oxandrolone, oxymetholone, stanozolol, testolactone, testosterone, testosterone cypionate, testosterone enanthate, testosterone propionate, and the like; or a progestin such as ethynodiol diacetate, gestodene, hydroxyprogesterone caproate, levonorgestrel, medroxyprogesterone acetate, megestrol acetate, norethindrone, norethindrone acetate, norethynodrel, norgestrel, progesterone, and pharmaceutically acceptable salts thereof.

81. The system of claim 66, wherein said drug is a locally active agent.

82. The system of claim 81, wherein the locally active agent is for use in the treatment of disorders of the skin selected from the group consisting of psoriasis, eczema, acne, nappy rash, other inflammatory disorders, bacterial infections, viral infections, fungal infections, anaphylactic conditions, malignancies and warts.

83. The system of claim 81, wherein the at least one therapeutic agent is selected from the group consisting of anesthetics, corticosteroids, antibacterial agents, antifungal agents or any therapeutically effective combination thereof.

84. The system of claim 81, wherein the at least one therapeutic agent is selected from the group consisting of tetracaine, benzocaine, lindocaine, hydrocortisone, beclomethasone diproprionate, clobetasol proprionate, fluticasone proprionate, ichthammol, lithium succinate, coal tar, dithranol, benzoyl peroxide, tretinoin, sulphur, vitamin D and derivatives thereof, framycetin, chlortetracycline hydrochloride, fusidic acid, clotrimazole, econazole, amorolfine and terbenafine, or any therapeutically effective combination thereof.

85. The system of claim 83, wherein said anesthetic is selected from the group consisting of bupivacaine, levo-bupivacaine, ropivacaine, benzocaine, dibucaine, procaine, chloroprocaine, prilocalne, mepivacaine, etidocaine, tetracaine, lidocaine, and xylocalne, as well as anesthetically active derivatives, analogs, isomers and mixtures thereof.

86. The system of claim 81, wherein the locally active agent is selected from the group consisting of antiviral agents (e.g., acyclovir and idoxuridine, etc.), antifungal agents (e.g., amphotericin B, clotrimazole, nystatin, ketoconazole, miconazole, butocouazole, haloprogin, etc.), antibiotic agents (penicillins, cephalosporins erythromycin, tetracycline, clindamycin, aminoglycosides, chloramphenicol, polymixin b, bacitracin, neomycin, gentamycin etc.), antiseptics (e.g., povidone-iodine, methylbenzethonium chloride, etc.), antiparasitics (e.g., lindane, anthralin, etc.) analgesic agents (e.g., methylsalicylate, salicylic acid, dyclonine, aloe vera etc.), local anesthetics (e.g., benzocaine, lidocaine, xylocalne, butamben picrate, etc.), anti-inflammatory agents (e.g., steroidal compounds such as dexamethasone, betamethasone, prednisone, prednisolone, triamcinolone, hydrocortisone, alclometasone, amcinonide, diflorasone, etc. as well as non-steroidal anti-inflammatories), anti-itch and irritation-reducing compounds (e.g., antihistamines such as diphenhydramine and psoriasis treatments) burn relief compounds (e.g., o-amino-p-toluenesulfonamide, monoacetate, etc.); depigmenting agents (e.g., monobenzone); and hormonal agents (e.g., oestriol).

87. The system of claim 66, wherein said therapeutic agent has non-local activity.

88. The system of claim 87, wherein said therapeutic agent is selected from the group consisting of vasodilators, active substances for the treatment of motion sickness, contraceptive agents, hormone replacement agents, painkillers, and smoking cessation aids, or any therapeutically effective combination thereof.

89. The system of claim 87, wherein said therapeutic agent is selected from the group consisting of nitroglycerin, scopolamine, estradiol, norethisterone, fentanyl and nicotine, or any therapeutically effective combination thereof.

90. The system of claim 66 wherein said carrier comprises a polymer.

91. The system of claim 90 wherein said polymer is selected from the group consisting of sodium alginate, gelatin, corn starch, gum tragacanth, methylcellulose, hydroxyethylcellulose, carboxymethylcellulose, xanthan gum, dextrin, carboxymethylstarch, polyvinyl alcohol, sodium polyacrylate, methoxyethylene-maleic anhydride copolymer, polyvinyl ether, polyvinylpyrrolidone,

92. The system of claim 66 wherein said carrier comprises a wax a fat, an oil or a combination thereof.

93. The system of claim 92 wherein said fat or oil is selected from the group consisting of beeswax, olive oil, cacao butter, sesame oil, soybean oil, camellia oil, peanut oil, beef fat, lard and lanolin.

94. The system of claim 66 wherein said carrier comprises white petrolatum.

95. The system of claim 66 wherein said carrier comprises a paraffin.

96. The system of claim 66 wherein said carrier comprises a higher fatty acid.

97. The system of claim 96 wherein said higher fatty acid is stearic acid.

98. The system of claim 66 wherein said carrier comprises a higher alcohol.

99. The system of claim 98 wherein said higher alcohol is selected from the group consisting of cetyl alcohol, stearyl alcohol and combinations thereof.

100. The system of claim 66 wherein said carrier comprises a polyethylene glycol.

101. The system of claim 66 wherein said carrier comprises water.

102. A method for topically administering a pharmaceutical formulation to the skin of a mammal, the method comprising,
(i) actuating a dosing device for topically administering a pharmaceutical formulation to the skin of a mammal, the device comprising:
a housing storing at least one unit dose of a pharmaceutical formulation comprising a drug incorporated with a pharmaceutically acceptable carrier suitable for topical application onto the skin of said mammal; an applicator adapted for topically administering a unit dose of the pharmaceutical formulation directly onto the skin; and an actuator capable of metering a single unit dose of the pharmaceutical formulation from a first position in which the unit dose is stored in the housing to a second position in which the single unit dose is external to the device on the applicator so that the single unit dose can be topically administered; and
(ii) applying the unit dose directly onto the skin of a mammal with the applicator.

103. The method for topically administering a pharmaceutical formulation of claim 102, further comprising actuating said device a second time and administering additional unit doses of said pharmaceutical formulation.

104. The method of claim 102 wherein said drug provides a local effect on the surface of the skin.

105. The method of claim 102 wherein said drug is absorbed and provides a local effect in the region of application.

106. The method of claim 102 wherein said drug is absorbed and provides a systemic effect.

107. A method of preparing a dosing system for topical delivery of a pharmaceutical formulation comprising:
(i) preparing at least one unit dose of a pharmaceutical preparation comprising a drug incorporated with a pharmaceutically acceptable carrier suitable for topical application onto the skin of said mammal; and
(ii) placing the at least one unit dose into a dosing device comprising a housing capable of storing at least one unit dose of a pharmaceutical formulation comprising a drug incoporated with a pharmaceutically acceptable carrier suitable for topical application onto the skin of said mammal; an applicator adapted for topically administering a unit dose of the pharmaceutical formulation directly onto the skin; and an actuator capable of metering a single unit dose of the pharmaceutical formulation from a first position in which the unit dose is stored in the housing to a second position in which the single unit dose is external to the device on the applicator so that the single unit dose can be topically administered

108. The device of claim 1 wherein the device is a unitary device.

109. The device of claim 1 wherein the actuator is flush with the surface of the device.

110. The device of claim 1 wherein the actuator is recessed from the surface of the device.

111. The device of claim 1 which can be submersed in water for at least 30 seconds without having the housing infiltrated with water.

112. The device of claim 1 wherein the housing is airtight.

113. The device of claim 1 wherein said housing is comprised of aluminum at the point of contact with the composition.

114. The device of claim 1 wherein said housing is comprised of plastic coated with aluminum at the point of contact with the composition.

115. The device of claim 41 wherein said counter is mechanical

116. The device of claim 41 wherein said counter is electronic.

117. The device of claim 1 comprising a visual aid to determine the number of unit doses remaining in the device.

118. The device of claim 117 wherein said visual aid is a transparent window to the inside of the housing.

119. The device of claim 1 which allows less than about 10% overage of the pharmaceutical composition.

120. The device of claim 1 which allows less than about 5% overage of the pharmaceutical composition.

121. The device of claim 1 which does not require overage of the pharmaceutical composition.

122. The method of claim 102 wherein said applying is self administration.

123. The method of claim 102 wherein said applying is by a caregiver to a patient.

124. The system of claim 66 wherein said drug comprises nitrogen oxide.
